(19) United States
(12) Patent Application Publication
(10) Pub. No.: US 2003/0170638 A1
(43) Pub. Date: Sep. 11, 2003

(54) Methods to determine genetic risk through analysis of very large families

(76) Inventors: Raymond L. White, Salt Lake City, UT (US); Richard Kerber, Salt Lake City, UT (US)

Correspondence Address: Alison B. Mohr, Parsons Behle & Latimer, Suite 1800, 201 South Main Street, Salt Lake City, UT, 84111-2218, US

(21) Appl. No.: 10/092,672

(22) Filed: Mar. 7, 2002

(51) Int. Cl. G06F 17/60; C12Q 1/68; G06F 19/00; G01N 33/48; G01N 33/50

(52) U.S. Cl. 435/6; 702/20; 705/3

(57) ABSTRACT

Methods are disclosed for predicting family and individual genetic risk of disease through the analysis of very large families (VLFs). A predetermined founder is identified. The definition of family is broadened to include about 100 or more decedents from the founder. The VLF can then be linked to a disease registry to determine if there is a significant excess of disease. This method can further identify individuals at risk for disease. The identified individuals and their immediate family members can then provide DNA samples. These samples can be used to identify the susceptibility gene.

### FIELD OF THE INVENTION

The present invention relates to the field of identifying genetic risk for disease, and in particular, identifying the function of genes.

### BACKGROUND OF THE INVENTION

Estimating familial genetic risk for common diseases is important in medical practice and research. Increased genetic risk of colon cancer, for example, can stimulate early and frequent screening by colonoscopy, greatly reducing the chances of developing a carcinoma. Colonoscopy is expensive, however, and identification of those at highest risk would provide its most cost-effective implementation. Furthermore, identification of sets of individuals carrying genetic susceptibility could help in identifying the genes and their variants that confer risk, leading to improved diagnostics and therapies.

A significant proportion of many of the major diseases that plague humanity is associated with genetic predisposition. Cancers, heart disease, asthma, stroke and diabetes are good examples. Generally 20 to 30% or more of the population disease burden is attributed to predisposing alleles (one of a series of possible alternative forms of a gene) of specific genes. In some cases, such as cancer or heart disease,, there are families that carry mutant alleles of specific genes that strongly predispose, such that inheritance of the mutant allele virtually guarantees that the cancer or cardiovascular disorder will appear. However, these families and individuals with such highly penetrant alleles account for generally less than 10% of the population burden of genetic predisposition. Therefore, more than 90% of the genes/alleles that predispose to common disease have not yet been identified.

In addition, there is good reason to expect that the penetrance of the alleles responsible for the majority of the population burden of genetic predisposition must only be moderate as most of the strong family clusterings of inherited predisposition can be explained by highly penetrant mutant alleles of known genes. For example, only 10 to 30% of carriers of these moderately penetrant mutant alleles might show the disease trait. In addition, it follows that in order for these more moderately penetrant gene/allele systems to account for the large population burden of disease, they must also be relatively frequent in the population.

There has been good success in identifying the strongly predisposing gene/allele systems. In many cases, family studies have provided mapping information that has led to positional cloning of the genes. Hundreds of such genes and their variants have been identified for hundreds of relatively rare genetic syndromes. Although there exists good evidence for the role of genetic inheritance in susceptibility to the common diseases, such as cancer, cardiovascular disease, inflammatory diseases and diabetes, only a few of the genes that confer this susceptibility have been identified. For example, there is good evidence that more than 30% of colon cancer occurs among individuals in association with a significant genetic risk. However, the syndromic cancer genes APC and HNPCC account for less than 3% of these cases.

Generally, in these types of cases the investigator starts with a small proband family (for example, a small family identified due to some unusual disease characteristic) and works back up through the pedigree, and then back down the branches looking for those branches with a telltale cluster of cases that will indicate transmission of the mutant gene/allele. Large pedigrees with many affected individuals can be ascertained this way. Such conventional family studies, however, have been largely unsuccessful in identifying large pedigrees and determining the chromosomal locations of the more frequent, moderately penetrant gene/alleles. It is very difficult to follow the inheritance of the mutant allele in a large pedigree when the penetrance is only moderate, as branches where the mutant allele has traveled may show up in only very few affected individuals.

Alternate approaches are now being suggested through comparison of the genetic make-up of large sets of affected individuals to large sets of matched controls. These approaches remain problematic, however, because of significant technical problems in identifying appropriate control populations and major potential statistical problems if many gene/allele systems are responsible for the predisposition.

### SUMMARY OF THE INVENTION

The present invention meets the above-described needs and others. Additional advantages and novel features of the invention will be set forth in the description that follows or may be learned by those skilled in the art through reading these materials or practicing the invention. The advantages of the invention may be achieved through the means recited in the attached claims.

### BRIEF DESCRIPTION OF THE DRAWINGS

The accompanying drawings illustrate preferred embodiments of the present invention and are a part of the specification. Together with the following description, the drawings demonstrate and explain, but in no way limit, the principles of the present invention.

FIG. 1 illustrates an embodiment of a method of identifying a VLF and determining the statistical significance of a VLF with an apparent excess of a disease.

FIG. 2 illustrates an embodiment of a method of identifying families and individuals at risk.

FIG. 3 illustrates an embodiment of a method of identifying identity-by-descent regions and the associated susceptibility gene.

### DETAILED DESCRIPTION OF THE PREFERRED EMBODIMENTS

Definitions

Founder: a starting/beginning person for descendant analysis. A founder may be without precedent ancestral information. A founding couple consists of two founders.

Family: limited to about three generations. Includes nuclear family.

Carrier: individual with a susceptibility gene that may or may not be expressed.

Very Large Family (VLF): about 100 or more descendants descending from the founder.

Disease: includes traits measured on a quantitative scale, for example, diabetes, cancer, heart disease, hypertension, and the like.

General Population Incidence of Disease: a calculated rate of disease occurrence among a defined set of individuals that may or may not include members of a very large family. For example, the State of Utah population versus a very large family population, wherein the State of Utah population is the general population.

Incidence of Disease: a calculated rate of disease occurrence among individuals. Incidence of disease includes burden of disease.

Coaggregation: the co-occurrence of traits within families that would ordinarily be considered distinct.

Identity-by-Descent: carrier of the same allele in the same marker locus due to inheritance through a common ancestor.

Identity-by-State: carrier of the same allele in the same marker locus due to chance or inheritance. Includes identity-by-descent.

Penetrance: a carrier's chance of having/expressing the disease.

Variant: an individual sequence that is different from an arbitrary standard type sequence. The difference may occur through deletion, base change, etc.

The traditional approach to ascertaining genetic risk is through the anecdotal “family history,” where an individual is asked whether he has any known relatives with cancer or, perhaps, other common diseases. In general, an individual may have knowledge of disease among his closest relatives, such as brothers, sisters, or parents. The individual will not, in general, have knowledge of the health status of more distant relatives, such as cousins, aunts, uncles, and will almost never know the health status of even more distant relatives such as second and third cousins.

In addition, an individual's knowledge of his or her own family history may be of little utility, as many of an individual's close relatives may be silent carriers that do not express the disease. It is well understood that most of the genetic risk carried in the population is due to genetic variants that have only a low to moderate “penetrance.” That is, a carrier of a susceptibility variant may have only a low to moderate chance of expressing the disease. The family history, therefore, may not reveal that the individual carries a genetic susceptibility and consequently is at much higher than average risk of the disease.

An embodiment of the present invention differs dramatically in that instead of building a family history from the “inside out” as in the traditional approach, the family history is developed from the “outside in.” The “outside in” approach defines the family to include many more distant relatives. One embodiment of the present invention broadens the traditional idea of family by taking advantage of a computerized genealogical database. However, alternative methods of broadening the family by identifying distant relatives would be appreciated by one of skill in the art.

The “outside in” approach results in “Very Large Families” (VLFs) that are comprised of the descendants of a founder or founding couple, generally, consisting of about 100 or more family members. The founder is identified by virtue of the fact that the founder will have more descendants showing the disease than other founders who did not have a predisposing allele for the disease. VLFs identified from population-based genealogical databases and linked to disease registries, allow better estimates of personal risk of susceptibility of individuals to disease and improve the process of discovering the genetic variants that predispose individuals to disease. It should be appreciated by one of skill in the art that significant VLFs may be identified by other means of obtaining and linking health status information or medical records to descendants of a founder. This new approach insures that the variants discovered will explain much of the overall population burden of genetic susceptibility due to the population-wide scan.

A method of the invention includes the identification of VLFs carrying genetic variants that are predisposed to common diseases. The approach circumvents the problem of stepwise development of large families by redefining the idea of “family” to include not just immediate or close relatives, but a larger context of more distant relatives. The number of cases of disease, of a VLF with a founder carrying a susceptibility variant, would be greater than other families of similar size and structure. Therefore, the VLF with a carrier founder would be identifiable as a family carrying a common susceptibility allele. This approach relieves the need to expand a family by trying to track the inheritance of a susceptibility gene/allele through multiple branches based on appearance of the disease, and facilitates discovery of more distant links.

This change in the magnitude of the definition of “family” profoundly affects the ability to look at the genetics of genes underlying susceptibility to cancer as well as many other common diseases. The change is in some ways analogous to the change in mRNA transcript profiling that results in going from 200 cDNAs spotted on a filter to 5,000 cDNAs spotted on a small glass slide. In addition, the susceptibility of a family or individual to virtually any other phenotypic characteristic or quantitative metabolic trait may also be ascertained using VLFs.

A second valuable aspect of the VLF approach is that a single VLF will carry most of its susceptibility through transmission of only a single gene carrying the single variant brought in by the carrier founder. This reduction in genetic complexity makes the statistical analysis much more powerful, as the majority of the individuals with a specific disease will share the same variant of the same gene.

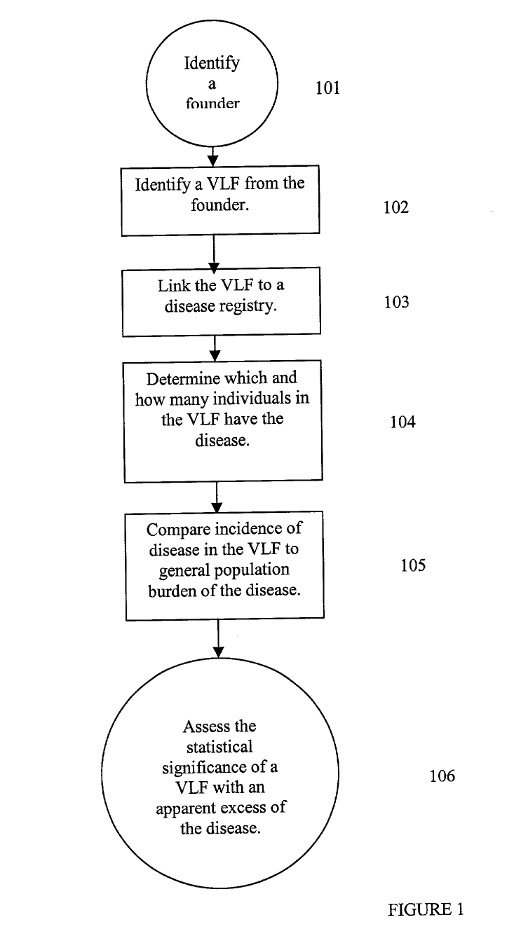

A method for identifying significant VLFs is illustrated in FIG. 1. First, the contributing founders are identified 101. This can be done by starting with a subpopulation of individuals in modern generations who are affected by a disease. In the case of colon cancer, for example, approximately 25% of these affected individuals will have colon cancer by virtue of having inherited a predisposing allele of a specific gene. By tracing the ancestors of each individual affected by colon cancer, it is found that the specific ancestors of the individuals, who have the cancer due to an inherited gene, will be identified significantly more frequently by this process than the ancestors of individuals whose cancer is not due to an inherited predisposition. That is, the descendants of a founder who is a carrier of a cancer variant will more frequently have cancer.

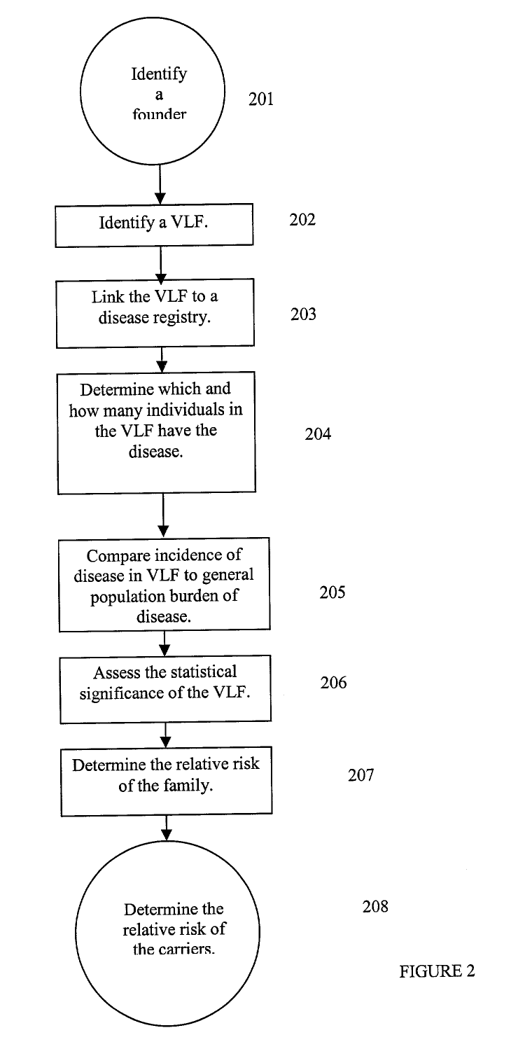

Second, a VLF is identified 102 using an identified founder. Third, the health status of the members of the VLF is determined by linking the VLF to a disease registry 103. Fourth, the number and distribution of disease cases is counted within the VLF 104. This is compared to expectation based on the number and distribution of disease cases predicted by the population average 105. The larger the number of disease cases, the greater the statistical significance 106. FIG. 2 further illustrates that family 207 and individual 208 risk numbers can then be calculated with confidence. Steps 201 through 206 parallel steps 101 through 106 of FIG. 1, respectively. Calculation of the relative likelihood of seeing the contribution of genetic risk due to moderate penetrance alleles transmitted by such distant founding relatives depends on the ability to create VLFs.

Defining the family in terms of this larger sample set now allows us to see footprints of the inheritance of a low to moderate penetrance allele. For example, if a transmitted allele has a 20% penetrance, then only 1 in 5 carriers will show the disease. Typically in a nuclear family setting there may be no more than a few relatives available for inspection. A relative affected by the disease may or may not be seen. On the other hand, if the family consists of 500 or more relatives, there may be as many as 50 or more carriers, in which case 10 or more individuals affected by the disease should be seen. This would be highly significant if the population expectation for the disease was only two affected individuals. One would therefore conclude that this is a high-risk family and individuals within this family may carry susceptibility to a specific disease.

Large families, in particular VLFs, have several important advantages over small families for the identification of disease-predisposing alleles in linkage/association studies. Chief among these are the relative efficiency of genetic linkage analyses in large families (in terms of information gained per genotype), and resistance to problems caused by locus (and allele) heterogeneity. The possibility of multiple genes, each able to confer susceptibility to the same disease, confounds linkage studies with sets of small families. For any given marker only a subset of the families will contribute to a statistical signal for a given chromosomal region. Some of the families will reflect the effects of one gene, other families will reflect the effects of a different gene, and still others will reflect the effects of a third gene, and so on. Attempting to add the statistical signals together from such a heterogeneous collection yields only very weak signals, localized only to quite broad chromosomal regions, making gene identification extremely difficult.

The possibility of multiple alleles, each capable of conferring susceptibility, likewise additionally confounds association studies within populations of unrelated individuals, as for a given marker only a subset of individuals will contribute to an association signal. However, analysis of individual VLFs that are large enough to contribute a significant linkage or association signal escapes these difficulties in that only a single allele of a single gene is likely to confer susceptibility to members of the same family.

A disadvantage of the traditional large family v. VLF studies has been the difficulty of identifying and sampling large families showing a consistent phenotype. VLFs identified from genealogical databases, however, are relatively easy to find, yet have all the advantages of traditional large families.

Large families, in particular VLFs, have greater power for studying most disease predisposition syndromes in that distant relatives have less chance of sharing alleles due to chance than more closely related individuals. Unaffected individuals will share chromosomal segments based only on chance segregation during meiosis-the more closely related, the greater this chance of allele sharing. For example, two siblings will carry half of their chromosome segments in common. However, when the same disease due to a genetic susceptibility allele affects both, they will almost always carry in common the chromosome segment that contains the susceptibility allele.

More distantly related unaffected relatives become increasingly unlikely to share a common chromosome segment because the chance of sharing a chromosome region due to inheritance from the common ancestor decreases by half at each generation. However, when the same disease due to a genetic susceptibility allele affects both, they will much more frequently carry in common the chromosome segment that carries the susceptibility allele. Thus, the observation of distant relatives affected with the same disease also sharing an allele (especially an infrequent allele) of a genetic marker locus provides evidence that the gene carrying the susceptibility allele lies on the same chromosomal segment as the genetic marker.

Moreover, because of genetic recombination at each generation, the length of a chromosomal segment shared among distant relatives is shorter on average than that shared among close relatives. This means that when such excess allele sharing is observed among distant relatives, the common chromosomal segment will be shorter, and contain fewer genes. This is important, as each gene found within the common chromosomal region becomes a candidate for the disease gene and must be carefully examined for mutations. A smaller common chromosomal segment means fewer candidate genes and less work in sorting through to find the disease gene. For example, a 10 megabase chromosome segment is likely to carry 100 genes, while a 1 megabase chromosome segment is likely to carry only 10 genes.

In addition, by examining VLF data it may be found that the familial risk applies to more than one disease outcome coaggregating in the same family. Although many genetic syndromes predisposing individuals to complex diseases are marked by several possible disease outcomes, e.g. breast and ovarian cancers resulting from BRCAI mutations, colon and endometrial cancers resulting from MSH2 or MLH1 mutations, . . . , etc., the traditional approach to identification of kindred relies on the identification of clusters of close relatives with a single disease. Clusters of relatives with different diseases appear to be sporadic cases when viewed from this limited perspective. If, however, a set of hundreds or thousands of relatives can be assessed for disease outcome, statistically significant patterns of association between diseases can be assessed using objective epidemiological criteria. This will allow alleles that confer susceptibility to each of several diseases to be identified more easily.

Estimation of the risk of an individual within a VLF is an important clinical application. The number and distribution of disease cases would provide a strong basis for more accurate estimations of individual risk than is presently available. This information would, for example, lead to eligibility of the individual for more intensive cancer screening programs. In addition, by examining VLF data it may be found that the familial risk applies to more than one disease. Such information would be very important in a clinical setting, as the screening protocol would need to encompass each of the diseases to which an individual is susceptible.

An important research application is the identification of the gene and its variant responsible for the genetic susceptibility segregating in the family. Indeed, the ascertainment of VLFs is expected to be an important tool in the identification of the genes and their variants that confer susceptibility within the population.

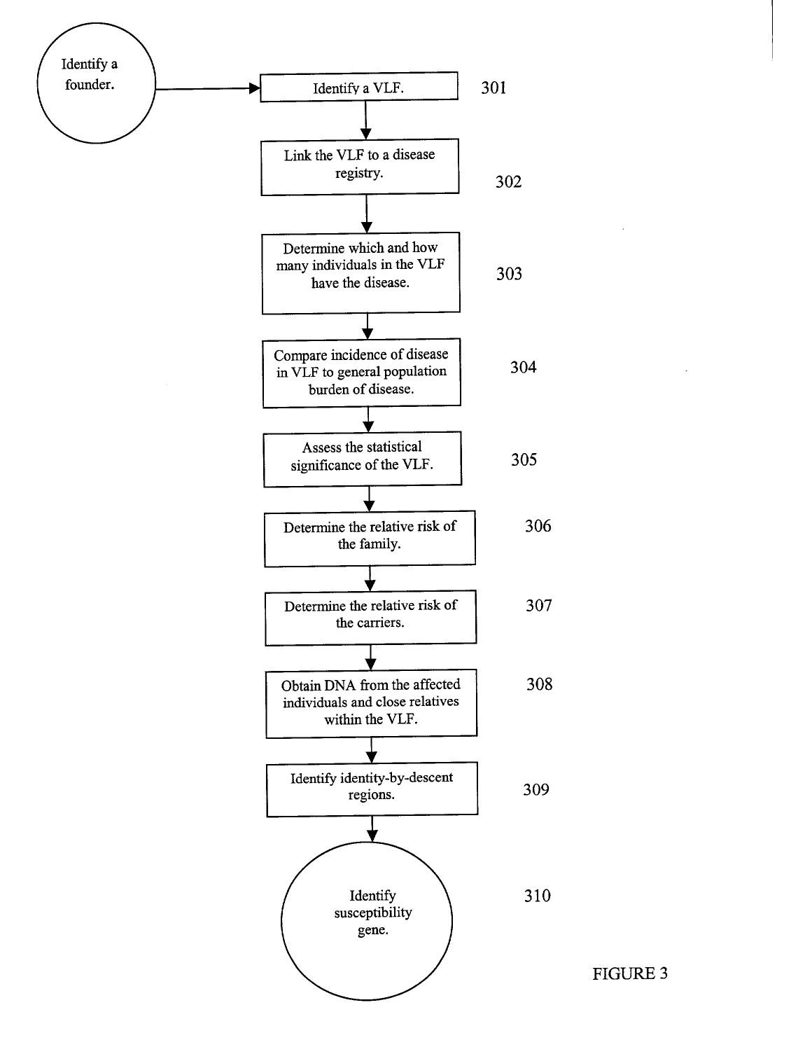

VLF analysis provides a method for the identification of the chromosomal location of the susceptibility gene as illustrated in FIG. 3. Steps 301 through 308 of FIG. 3 parallel steps 201 through 208 of FIG. 2, respectively. The method depicted in FIG. 3 includes obtaining DNA samples from affected individuals and their close relatives 309. Affected individuals who have inherited a susceptibility gene/allele from one of the two founders of the VLF will share a chromosomal region carrying the susceptibility gene/allele that is identical-by-descent. Association with single alleles of genetic markers that fall within the identical-by-descent region will identify the region 310. Specifically, the identical-by-descent chromosomes will each carry the same allele of markers that are physically nearby the susceptibility gene. The identity-by-descent region location will lead to the identification of the susceptibility gene 311.

The size of this identical-by-descent region is expected to vary over a wide range with an average size of 5 centiMorgans (megabases) to 15 centiMorgans (megabases) among affected individuals sharing an identical-by-descent region separated by 6 generations in the VLF. This is an important number as it determines how dense the genetic marker set must be.

For example, in one embodiment of the present invention, DNA samples from affected individuals in a VLF for which a moderate penetrance colon cancer gene/allele has been identified, were experimentally tested. The size of the chromosome segment inherited identical-by-descent between individuals is often greater than 12 megabases. However, the minimum region of overlap among 19 individuals from the VLF was between 7 megabases and 11 megabases. Therefore, a set of less than 1,000 well-spaced genetic markers will detect regions of identity-by-descent carried in association with the disease diagnosis.

Initial scans of family members with a high probability of carrying an identity-by-descent genetic marker in association with disease susceptibility may yield several regions where there is a marker showing increased allele sharing across the family members. One possibility is that the excess allele sharing identity-by-descent is due to chance in regions not associated with the disease susceptibility. Although this should happen only 0.1% of the time between any pair of individuals separated by 6 generations, 1,000 markers are used yielding an expectation that allele sharing by identity-by-descent, not related to disease susceptibility, on average will be once for each pair-wise comparison. However, in general, there are several such independent comparisons within each VLF. It becomes highly unlikely that the existence of three identity-by-descent regions among three affected individuals, for a region not associated with the disease susceptibility allele, would be seen.

A more concerning and frequent component is allele sharing among affected individuals where the sharing is due to identity-by-state. That is, alleles that look the same but have not come into the family through the founding pair. For example, very good marker systems might have a number of alleles each represented at 10% frequency in the unselected population. The chance that two individuals each share at least one such an allele identical-by-state would be 0.04+(0.01*0.2*2=0.004)+0.0001=0.0441. With 1,000 markers our expectation is 44 identity-by-state pairings. However, as the numbers of affected individuals increases, the allele sharing due to chance combinations of identity-by-state alleles will decrease rapidly. For three affected individuals for example, the likelihood of identity-by-state decreases to about 12% and so on. The likelihood of identity-by-state is quite small with 10 -20 affected individuals.

However, the likelihood of identity-by-state allele sharing can be made as small as desired by reducing the frequency of the associated haplotype. This reduction is readily accomplished by creating simple tandem repeat (STR) haplotypes covering each of the marker regions showing excess allele sharing. Each individual haplotype thus becomes an allele in a new marker system; with marker spacing every 200 kb. For example, complete linkage equilibrium among the STRs should be seen, such that each haplotype comprised of five such STR markers, each of which with five equally frequent alleles, would have a frequency of 0.00032 and the likelihood of two individuals sharing such an allele identity-by-state is approximately 1/100,000. Only one haplotype should survive this test, the one that is closely associated with the disease, thus providing a unique localization for the disease gene.

An embodiment of the present invention has application in gene identification research. The 5 mb to 15 mb size of the chromosomal region expected to be identified in a VLF is much smaller than the 30mb regions resolved by conventional small family studies for common disorders. However, in the endgame of identifying the specific gene within the region that carries the variants associated with susceptibility, each gene in the region becomes a candidate. Due to an expectation of an average of 10 to 20 genes per megabase, there remains a large number of genes to be identified within the region and scanned for the presence of variants that might cause susceptibility.

It is also anticipated that several families will show association between their cancer susceptibility and the same chromosomal region. The size of the region may be reduced by looking at the overlap in chromosomal identity-by-descent among the several families. Furthermore, within each candidate region, finding genes of known function is anticipated, a few that will show characteristics expected of a disease susceptibility gene, such as a role in DNA repair. These genes become candidates by virtue of their function as well as their location and, thus further limiting the number of genes for which detailed examination will be required. This approach is thus not only reasonable in principle, but should provide a highly practical approach to the challenging problem of mapping and identifying the genes and their variants associated with susceptibility to common diseases.

### EXAMPLES

### Example 1

Families from a computerized genealogical database, the Utah Population Database (UPDB), of about 500 to about 10,000 or more, were scanned for excess numbers of specific cancers by linking to a database of cancer cases (the Utah Cancer Registry) and determining whether the number and distribution of cases in the VLF differs from chance expectations. The UPDB currently contains records of about 1.7 million individuals born between 1800 and the present, spanning 1-9 generations. Of these individuals, about 660,000 have been at risk for cancer as recorded by the Utah Cancer Registry between 1966 and the present. This number is, in part, so large due to the increasing population of Utah with each generation. According to the example below, there are VLFs within these databases with an excess of specific cancers. Furthermore, in a number of such instances the VLFs were found to have an excess of more than one kind of disease.

To summarize familial risks, estimates were prepared for the genetic relative risk for each founder and the exact probability that any observed excess of disease among the descendants of the founder was the result of chance. In simulation studies, the combination of these measures (high relative risk, low probability) has proved to reliably identify kindred in which a disease-predisposing allele is segregating. The probability that some number of disease cases is observed among the descendants of a founder, given some number of person-years of risk among his or her descendants, is

$$p ( x ) = \frac{{λ}^{x} {ⅇ}^{- λ}}{x !}$$

where x is the number of diseases observed and λ is the number expected given the total person time experienced in each of some number of risk strata based on age and sex. Considering only situations in which the observed number of cases (x) is greater than the expected number (λ), the probability of x or more cases being observed in a given family is

$$p ( X \geq x ) = 1 - [ {\sum }_{j = 0}^{x - 1} \frac{{λ}^{j} {ⅇ}^{- λ}}{j !} ] .$$

The occurrence of a complex disease with the incidence characteristics of colon cancer (late onset, similar risks for males and females, lifetime risk around 5%) in a set of 660,000 people at risk for cancer drawn from the UPDB, was simulated. A genetic predisposition syndrome with characteristics derived from analyses of colon cancer in the UPDB was simulated, with a predisposing allele frequency of 4% and a relative risk to carriers of 9.0. The high allele frequency makes identifying particular founders relatively difficult, because a high proportion of marry-ins in any given kindred will be expected to carry the predisposing mutation.

About 44,000 founders contributed genes to the cohort of individuals at risk. The techniques described above were used to identify the founders most likely to have contributed predisposing mutations to the descendant population. The table below summarizes the results.

TABLE 1

| | |
|---|---|
| Simulated Data | Colorectal |

| | | Positive | | Cancer Data |
|---|---|---|---|---|
| Threshold | Observed/ | Predictive | Relative | Observed/ |
| p-value | Expected | Value | Enrichment | Expected |
| 0.01 | 1.23 | 54% | 4.54 | 4.11 |
| 0.001 | 3.01 | 68.9% | 5.79 | 11.63 |
| 0.0001 | 15.06 | 80.3% | 6.74 | 27.93 |
| 0.00005 | 30.12 | 74.3% | 6.24 | 55.87 |

The ratio of observed to expected families increased as the expected probability decreased, clearly indicating the degree of excess familial clustering associated with the simulated high-risk genotype. Positive predictive values (the proportion of true positives out of the set of test positives) increased steadily until the 0.0001 threshold, and then appeared to plateau. The relative enrichment increased as a function of positive predictive value. It has been found that the selection of kindred with an excess risk of disease among descendants such that the p-value calculated above is less than 0.01 substantially improves the ability to identify families that carry predisposing alleles.

### Example 2

In simulation studies, the relative risk of disease was calculated for large families and VLFs.

Let xₖ be the number of cases observed among relatives of degree k, and λₖ be the number expected among non-carriers given the amount of person-time in a set of age-and sex-specific risk strata. If RR₀ represents the relative risk to carriers of a dominant predisposing allele, the risk to relatives of the carrier of degree k is given by:

$${RR}_{k} = {2}^{- k} ( {RR}_{0} ) + ( 1 - {2}^{- k} ) = 1 + \frac{( {RR}_{0} - 1 )}{{2}^{k}}$$

assuming no inbreeding and random mating. Thus, the probability of the observed counts of cases X₁, X₂, . . . ,X_{κ}over the entire set of relatives of a proband, given RR₀, is

$$L = {\prod }_{i = 1}^{K} \frac{{( {RR}_{k} {λ}_{k} )}^{{x}_{k}} {ⅇ}^{- ( {RR}_{k} {λ}_{k} )}}{{x}_{k} !} .$$

This likelihood (L) can then be used to obtain maximum likelihood estimates of RR₀, the relative risk to carriers. With appropriate stratification, the assumption that carriers have proportional risks in all risk strata can be relaxed and/or tested.

In Table 2, the mean and median values of RR₀ were compared for carriers and non-carriers of the susceptibility gene in the simulated data described above. The true value of RR₀ is 9.0 for carriers and 1.0 for non-carriers. The columns in Table 2 compare carrier risk estimates calculated from all families in UPDB to those calculated on large families (with at least one expected case of colorectal cancer among descendants) and even larger families (with at least five expected cases). The “Large Families” have an average of over 600 members as an average of 662 descendants is required for one case of colorectal cancer to be expected. The VLFs have an average of over 3,000 members. Table 2 shows that as the size of the families grows, the estimated carrier relative risks approach the true values for both groups.

TABLE 2

| | Large Families | Very Large |
|---|---|---|
| | (Total Expected | Families (Total |
| All Families | 1) | Expected 5) |

| | Median | Mean | Median | Mean | Median | Mean |
|---|---|---|---|---|---|---|
| Carriers | 0.0 | 11.8 | 5.7 | 7.8 | 6.0 | 7.1 |
| Non- | 0.0 | 5.3 | 0.1 | 2.7 | 0.9 | 2.5 |
| carriers | | | | | | |

The preceding description has been presented only to illustrate and describe the invention. It is not intended to be exhaustive or to limit the invention to any precise form disclosed. Many modifications and variations are possible in light of the above teaching. Some, although not all, alternative embodiments are described. The preferred embodiment was chosen and described in order to best explain the principles of the invention and its practical application. The preceding description is intended to enable others skilled in the art to best utilize the invention in various embodiments and with various modifications as are suited to the particular use contemplated. It is intended that the scope of the invention be defined by the following claims.

What is claimed is:

1. A method comprising:
a. selecting at least one founder;
b. identifying a very large family from the founder;
c. linking the very large family to a disease database;
d. determining an incidence of disease by calculating which and how many individuals within the very large family have the disease;
e. comparing the incidence of disease in the very large family to a general population incidence of disease; and
f. assessing a statistical significance of the disease incidence in the very large family.

2. A method as in claim 1, comprising:
determining a relative risk of incidence of disease for the very large family.

3. A method as in claim 2, comprising:
determining a relative risk of incidence of disease for an individual within the very large family.

4. A method as in claim 1, comprising:
obtaining DNA samples from individuals with disease and their family within the very large family.

5. A method as in claim 4 comprising:
identifying identity-by-descent regions within the DNA samples.

6. A method as in claim 5, comprising:
identifying a susceptibility gene within the identity-by-descent regions.

7. A method comprising:
determining a relative risk of incidence of disease for a very large family.

8. A method as in claim 7, comprising:
determining a relative risk of incidence of disease for an individual within the very large family.

9. A method comprising:
a. obtaining DNA samples from individuals with disease and their family within a very large family; and
b. identifying identity-by-descent regions within the DNA samples.

10. A method as in claim 9, comprising:
identifying a susceptibility gene within the identity-by-descent regions.
